(19) United States
(12) Patent Application Publication
(10) Pub. No.: US 2025/0213382 A1
(43) Pub. Date: Jul. 3, 2025

(54) MODULAR ADJUSTABLE CAST STIRRUP

(71) Applicant: Sarah Alger, Toronto (CA)

(72) Inventor: Sarah Alger, Toronto (CA)

(21) Appl. No.: 18/851,310

(22) PCT Filed: Mar. 31, 2023
(86) PCT No.: PCT/CA2023/050442

§ 371 Date: Sep. 26, 2024

Related U.S. Application Data
(60) Provisional application No. 63/326,106, filed on Mar. 31, 2022.

(51) Int. Cl. A61F 5/058

(52) U.S. Cl. CPC A61F 5/0585

(57) ABSTRACT

The present invention provides an adjustable cast stirrup device comprising modular components that are adjustable during assembly to vary the width and length of the device to provide a customizable fit on a patient. Also provided is a kit comprising the components for forming the adjustable cast stirrup, as well as tools for assembly and instructions for use.

### FIELD OF THE INVENTION

The present invention pertains to the field of medical devices, and in particular to devices for the treatment of foot injuries.

### BACKGROUND

Patients that require custom cast stirrups for diabetic foot ulcers, foot surgery recovery or foot and ankle trauma are typically required to be measured for the device and have it custom made. This takes a considerable amount of time, and as such, the device is currently only available for those that plan well in advance to have it and require it to be custom made. For those patients requiring the device to be built into their cast at the time of injury or surgery, this option is not available.

Tamir et al. describe the use of a such a custom-made metal stirrup in the treatment of (Foot & Ankle International, Vol. 28, No. 10, pages 1048-1052 October 2007).

Due to the inconvenience associated with the amount of time required to manufacture a custom cast stirrup, there is a need for a cast stirrup that can be prepared on site and at the time of injury or treatment, without sacrificing the benefit of customized fit on the patient.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide a modular adjustable cast stirrup. In accordance with an aspect of the present invention, there is provided a modular adjustable cast stirrup device comprising: a first bracket member and a second bracket member, each of the bracket members being formed from a rigid strip having a horizontally oriented first end portion, a middle portion extending vertically upward from the first end portion, a second end portion, and an inwardly angled transition portion extending between the middle portion and the second end portion; a first adjustable side arm member and a second adjustable side arm member, each of the adjustable side arm members comprising at least one leg clamp located on an inner side of each respective side arm member, wherein each of the adjustable side arm members is configured for removable and adjustable attachment to the second end portion of a respective bracket member; a horizontally oriented foot plate member being configured for attachment to the first end portion of each said bracket member; wherein the relative position of the bracket member and the side arm member is adjustable to vary the length and/or height of the cast stirrup, and wherein the relative position of the bracket member and the foot plate is adjustable to vary the width of the cast stirrup.

In accordance with another aspect of the present invention, there is provided a process for assembling the cast stirrup of the present invention, comprising the steps of: affixing the foot plate to the bracket members by aligning the foot plate the horizontally oriented first end portion of the bracket members to provide a desired stirrup width and attaching the foot plate and bracket members using screw attachments; and affixing the side arm members to the upper end of the bracket members by aligning the side arm members with the second end portion of the bracket members to provide a desired height of the assembled cast stirrup and attaching the side arm members and bracket members using screw attachments.

In accordance with another aspect of the present invention, there is provided a process for installing an assembled cast stirrup on a patient's casted leg, comprising the steps of: placing the assembled stirrup over the patient's casted leg; aligning the stirrup with a longitudinal axis of the patient's casted leg; positioning the stirrup about 2 inches below a base of the cast and ensuring the base of the cast is parallel to the base of the stirrup; applying a thin layer of soft roll over an upper portion of the stirrup including the leg clamps and side arm members; applying a fiberglass cast material overwrap around the upper portion of the stirrup and cast to secure the stirrup in place; and allowing the fiberglass overwrap to set.

In accordance with another aspect of the present invention, there is provided a kit for assembling a cast stirrup device comprising: two bracket members, each said bracket members having a horizontally oriented first end portion, a middle portion extending vertically upward from the first end portion, a second end portion, and an inwardly angled transition portion extending between the middle portion and the second end portion; two adjustable side arm members, each of the adjustable side arm members comprising at least one leg clamp located on an inner side of each respective side arm member, wherein each of the adjustable side arm members is configured for removable and adjustable attachment to the second end portion of a respective bracket member; a foot plate member, the foot plate member being configured for removable and adjustable attachment to the first end portion of each said bracket member; a plurality of screw attachments; instructions for assembly; and optional tools for assembly.

### BRIEF DESCRIPTION OF THE FIGURES

FIG. 1 is a photograph of a prior art cast stirrup.

FIG. 2 is a perspective view of a cast stirrup, in accordance with one embodiment of the present invention.

FIG. 3 is an exploded perspective view of the components of a cast stirrup, in accordance with one embodiment of the present invention.

FIG. 4 is a cross sectional view of a cast stirrup, in accordance with one embodiment of the present invention.

FIG. 5 is a perspective view of a cast stirrup, in accordance with one embodiment of the present invention.

FIG. 6 is a perspective view of the cast stirrup of FIG. 5, showing sliding adjustment of the side arm member.

FIG. 7 is a perspective view of one stage of the assembly process of cast stirrup and cast assembly, in accordance with one embodiment of the present invention.

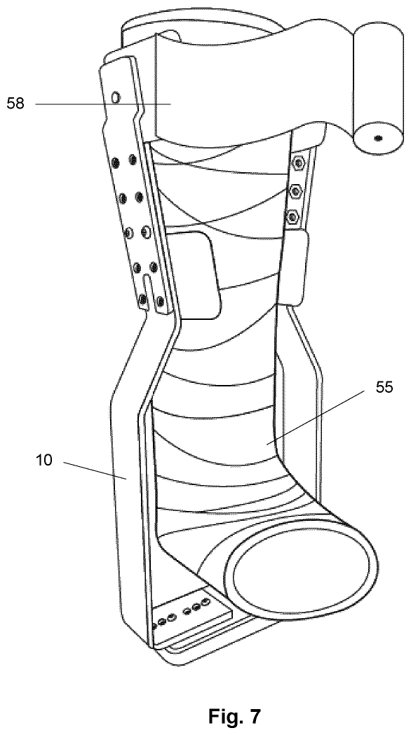

FIG. 8 is a perspective view of one stage of the assembly process of cast stirrup and cast assembly, in accordance with one embodiment of the present invention.

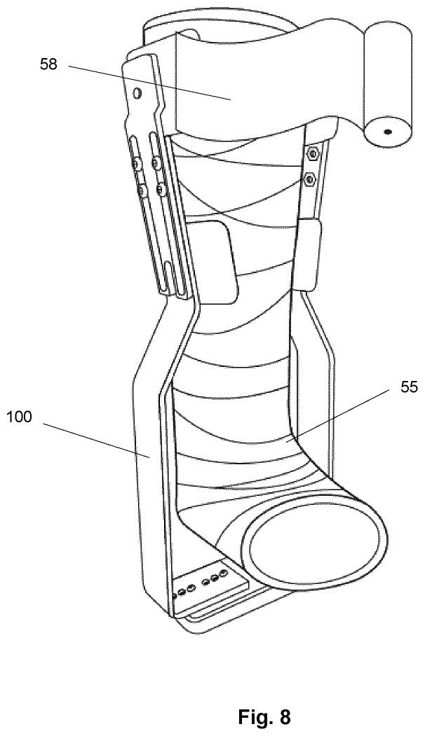

FIG. 9 is a perspective view of a cast stirrup and cast assembly, in accordance with one embodiment of the present invention.

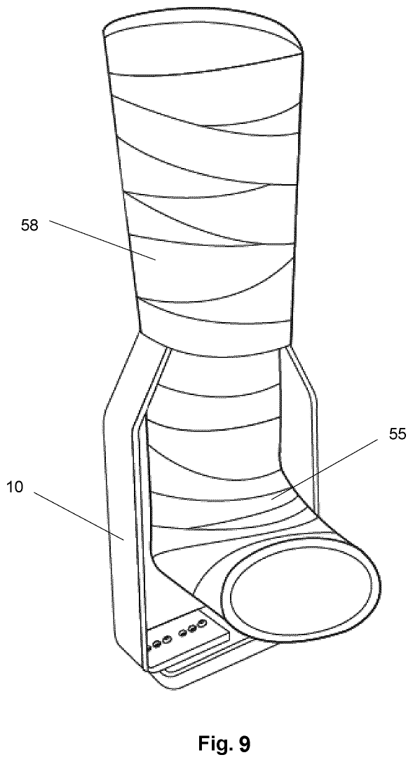

### DETAILED DESCRIPTION OF THE INVENTION

Unless defined otherwise, all technical and scientific terms used herein have the same meaning as commonly understood by one of ordinary skill in the art to which this invention belongs.

As used herein, the term “about” refers to a +/−10% variation from the nominal value. It is to be understood that such a variation is always included in a given value provided herein, whether or not it is specifically referred to.

The present invention provides a customizable cast stirrup that is adjustable for height and/or width to provide a patient with a readily assembled, custom fit stirrup. The stirrup of the present invention may be provided as a kit of parts that can be assembled and adjusted for customized fit on the patient's foot straight out of the box.

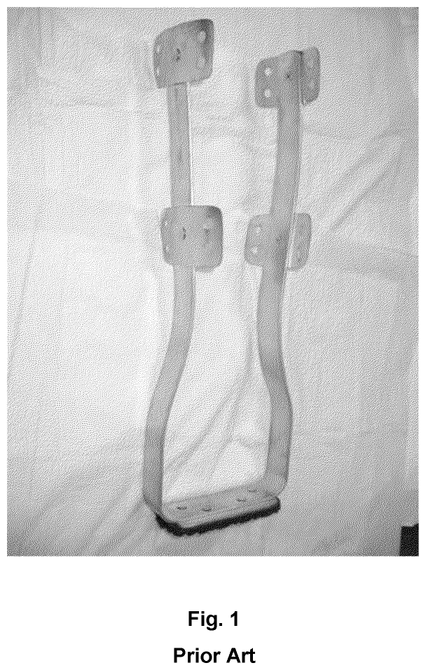

By contrast, prior art stirrups, being formed by shaping a single strip of metal according to measurements of the patient's foot and leg (such as is shown in FIG. 1), require crafting by a specialist, which necessarily leads to a delay between obtaining measurements of the patient's leg and the provision and fitting of the final cast and stirrup on the patient.

The customizable stirrup of the present invention therefore allows patients to be fitted with into the cast stirrup immediately without having to wait for the time typically required to complete a custom-made device formed from a single piece.

It is therefore possible to keep a selection of the component parts in stock, either individually or as a kit of parts, which can readily be selected on demand and adjusted to the required size of the patient. This ensures, for example, that diabetic patients get an offloading cast stirrup in a timely fashion to promote a higher success rate of wound closure reducing the risk of amputation. This also ensures that a suitable cast stirrup is readily available for trauma patients requiring foot and ankle surgery.

As such, more patients may be eligible for treatment using the stirrup of the present invention, since no advance planning or time to carry out measurement for the design and manufacture of a custom device are required. The present invention can therefore be expected to result in higher success rate for recovery from foot and ankle surgery.

In accordance with the present invention, the stirrup is used in conjunction with a foot and ankle cast. This stirrup allows a patient to walk with their casted leg without loading weight through the affected foot/leg.

In accordance with the present invention, the cast stirrup is adjustable for proper fit to the circumference and length of the leg.

Proper fit of the cast stirrup provides a contoured fit superior to the malleoli allowing for extra breadth of cast dressing with leg clamp flanges to accommodate the lower limb at the widest portion of the gastrocnemius/soleus complex and ending inferior to the fibular head so as not to cause compression on the common peroneal nerve.

In one embodiment, the stirrup is manufactured from any rigid, preferably lightweight, material. In a preferred embodiment, the stirrup is manufactured from aluminum.

The stirrup device has adjustable upper arms that are designed to be able to extend the height of the device based on the height of the patient and length of the leg. This is adjusted by changing the placement of the screw attachments in threaded holes. In one embodiment, there are five potential configurations for height.

The stirrup device is designed in a way that the lower portion of the stirrup device can also be made wider by changing the placement of the lower brackets on the foot plate. In one embodiment, this is adjusted by changing the placement of the screw attachments in threaded holes. In one embodiment, there are multiple width potential configurations.

As used in the present disclosure, the terms “screw attachments” and “screw” are used to describe any threaded attachment means such as a screw or a bolt which can be used to removably attached.

In one embodiment, the device has four leg clamp flanges that curve around the patient's cast. These flanges and upper arms of the device are fixed to the cast using an additional roll of fibreglass cast bandages. The base of the device has an aluminum foot plate that has a rubber sole, or foot pad, attached. In one embodiment, the sole or foot pad is affixed using contact cement.

In accordance with the present invention, the modular adjustable cast stirrup is formed from two bracket members, two adjustable side arm members, and a foot plate member.

The bracket members are each formed from a rigid strip of material pre-bent into a shape comprising a horizontally oriented first end portion, a middle portion extending vertically upward from the first end portion, a second end portion, and an angled transition portion extending inwardly from the middle portion to the second end portion. The adjustable side arm members are configured for removable and adjustable attachment to the second end portion of a respective bracket member.

In accordance with the present invention, the relative position of the bracket member and the side arm member may be adjusted to vary the overall length/height of the cast stirrup. In one embodiment, the adjustable side arm member is attached to the bracket member by screw attachment. In a further embodiment, the adjustable side arm member comprises a series of screw hole arrangements, each arrangement corresponding to a different stirrup length/height. In yet a further embodiment, where the side arm member is manufactured from aluminum, the screw holes are formed from a stainless steel threaded insert embedded in the aluminum arm member.

The foot plate member of the cast stirrup is configured to be attached to the first end portion of each bracket member. In accordance with the present invention, the relative position of the bracket member and the foot plate may be adjusted to vary the width of the cast stirrup. In one embodiment, the foot spacer plate is attached to the bracket member by screw attachment. In one embodiment, the first end portion of the bracket member comprises a series of screw hole arrangements, each arrangement corresponding to a different stirrup width. In another embodiment, the foot spacer plate has two parallel slots that are configured to receive screws for attachment to the foot plate, where the width of the stirrup can be adjusted by sliding the bracket members relative to the foot plate until the desired width is achieved.

In one embodiment, each of the adjustable side arm members comprises at least one leg clamp located on an inner side of each respective side arm member.

In one embodiment, each of the bracket members optionally comprises a leg clamp located on an inner side of the second end portion.

The leg clamps are located such that, when placed in contact with the cast and covered with further casting material, the stirrup is firmly and stably held in place, and will not move relative to the cast.

In one embodiment of the invention, there is provided a kit of parts comprising all components required for assembly of the cast stirrup, including bracket members, adjustable side arm members, and foot plate, as well as screws, tools for assembly, and instructions for use.

In a preferred embodiment, a fiberglass casting material is applied to the leg and foot to form the primary cast, the metal stirrup is placed against the primary cast, and the stirrup is then secured to the primary cast by wrapping with another layer of fiberglass casting material around the stirrup and primary cast.

In accordance with the present invention, the stirrup is assembled according to the following simple process steps. The L-shape bracket members are initially affixed to the foot plate by aligning screw holes on the foot plate with the screw holes on the L-shaped bracket members and inserting four screws to attach the foot plate to L-shape bracket. The position of the L-shaped brackets relative to the foot plate is selected to provide an overall stirrup width that allows the stirrup to fit comfortably over the patient's cast. Once the appropriate width has been selected, the screws are tightened with a hexagonal key or screwdriver.

Following assembly of the bottom portion of the stirrup, the side arm members are affixed to the upper end of the bracket members. The relative position of the side arm members relative to the upper portion of the bracket members is selected to provide an overall height that ensures the top of the assembled stirrup will sit about 2 inches (3 finger clearance or about 5 cm) below the top-line of the cast while also maintaining about 2 inches (about 5 cm) of clearance between the plantar surface of the cast and the foot plate. Once the appropriate height has been selected, the two side arm members are aligned with the L-shaped bracket members, screws are inserted and tightened with a hexagonal key or screwdriver to attach the side arm members to the bracket members.

In one embodiment, screw holes located on the two side arm members are aligned with the screw holes at the upper end of the bracket members to achieve the desired configuration. In another embodiment, the two side arm members each have two parallel slots which are aligned with screw holes at the upper end of the bracket members and which are configured to receive screws for attachment of the side arm members to the bracket member upon sliding adjustment until the desired configuration is achieved

Once the stirrup has been assembled, it is placed over the patient's cast while ensuring that the stirrup is aligned (in-line) with the longitudinal axis of the lower limb and rests about 2 inches (about 5 cm) below the plantar surface of the cast (to ensure the patient cannot heel-toe walk). Ensure the base of the cast is parallel to the base of the stirrup, and apply a thin layer of soft roll over the upper portion of the stirrup (including the leg clamps and side arm members) to ensure that the device can be readily removed for reuse should the patient require a cast change. After application of the soft roll layer, wrap a layer of fiberglass cast material around the upper portion of the cast and stirrup to secure the stirrup in place (starting from the topline of the cast). Reconfirm alignment of the stirrup relative to the longitudinal axis of the leg before the fiberglass wrap sets, and ensure the fiberglass has set completely before allowing patient to bear weight on their leg and stirrup. If a cast change is required, the stirrup can be easily removed by cutting the external fiberglass wrap to release the stirrup.

If necessary, the curvature of the leg clamps and the shape of the bracket members can be adjusted or bent using bending irons or against a firm surface to accommodate a leg that is more cylindrical or conical in shape.

The invention will now be described with reference to the figures. It will be understood that the following description is intended to describe embodiments of the invention and are not intended to limit the invention in any way.

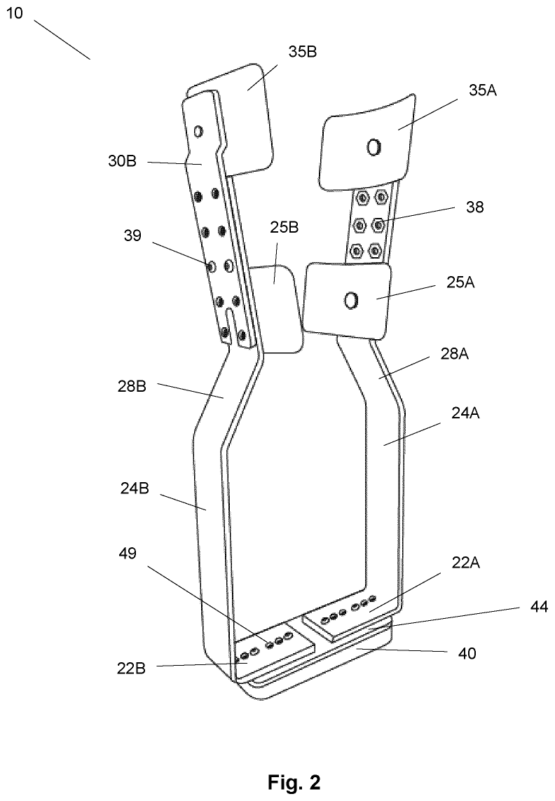

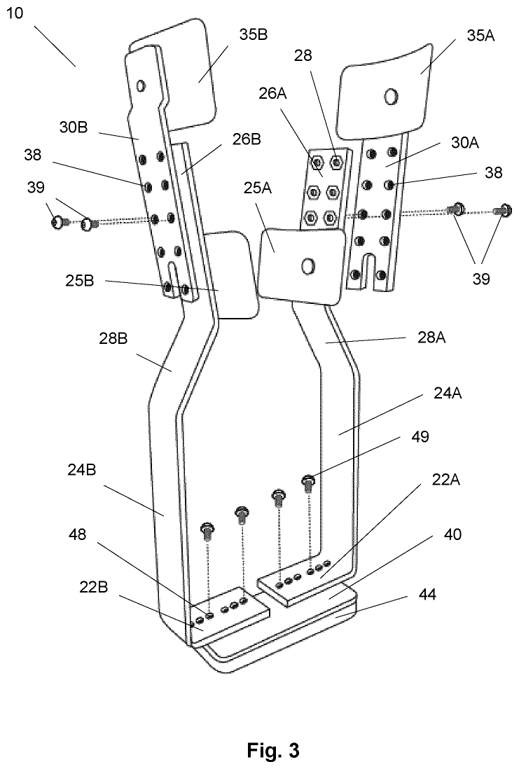

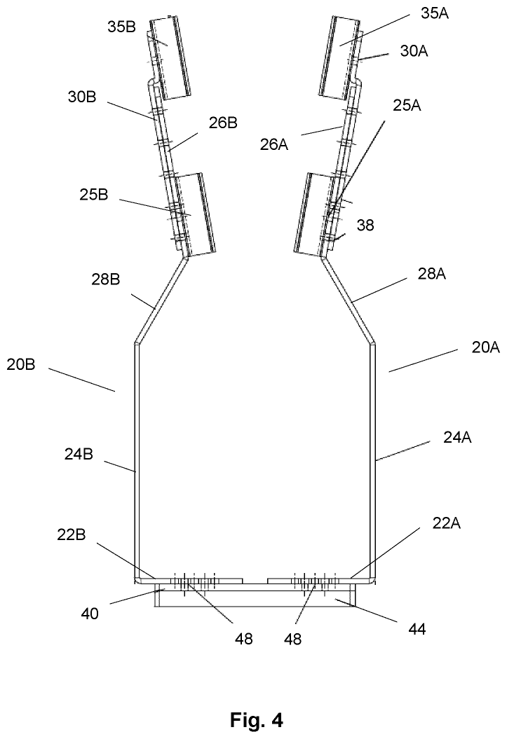

As shown in FIGS. 2 to 4, cast stirrup 10 is formed from two bracket members 20A and 20B, each having respective horizontally oriented first end portions 22A and 22B, middle portions 24A and 24B extending upwardly therefrom, and inwardly angled transition portions 28A and 28B extending from the middle portions 24A and 24B to second end portions 26A and 26B.

First end portions 22A and 22B are each attached to a horizontally oriented foot plate 40 using screws 49. First end portions 22A and 22B each comprise a series of screw hole arrangements 48 corresponding to screw holes (not shown) on foot plate 40, wherein each arrangement corresponds to a different stirrup width, such that the relative position of bracket members 20A and 20B and foot plate 40 are adjustable to vary the width of cast stirrup 10.

Second end portions 26A and 26B are adapted for attachment to adjustable side arm members 30A and 30B, respectively by screw attachment 39. Adjustable side arm members 30A and 30B each comprise a series of screw hole arrangements 38, corresponding to screw holes 28 of second end portions 26A and 26Bon the bracket members configured to obtain different overall stirrup lengths/heights as required.

Adjustable side arm members 30A and 30B each further comprise respective upper leg clamps 35A and 35B. In a similar manner, second end portions 26A and 26B of bracket members 20A and 20B also comprise respective lower leg clamps 25A and 25B.

Foot plate 40 further comprises foot pad 44 to provide cushioning and grip on floor surfaces.

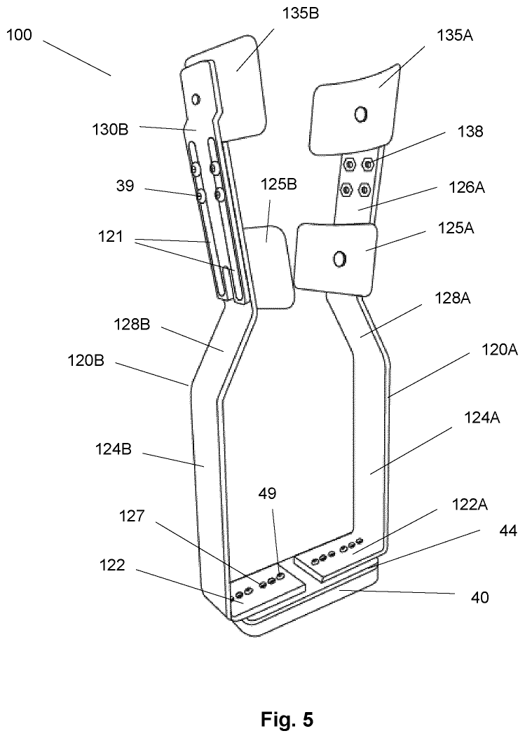

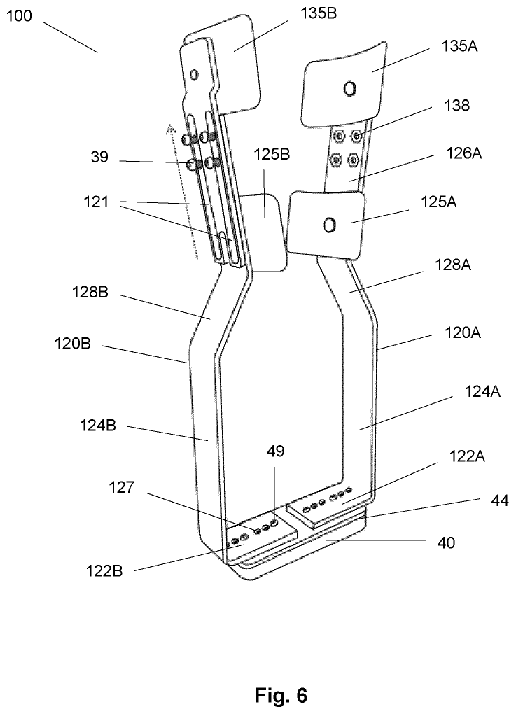

FIGS. 5 and 6 depict an alternative configuration of a cast stirrup which employs slots on the side arm members to facilitate adjustment of the overall length/height of the stirrup. As shown, cast stirrup 100 is formed from two bracket members 120A and 120B, each having respective horizontally oriented first end portions 122A and 122B, middle portions 124A and 124B extending upwardly therefrom, and inwardly angled transition portions 128A and 128B extending from the middle portions 124A and 124B to second end portions 126A and 126B.

First end portions 122A and 122B are each attached to a horizontally oriented foot plate 40 using screws 49. First end portions 122A and 122B each comprise a series of corresponding screw hole arrangements 48 corresponding to screw holes (not shown) on foot plate 40, wherein each arrangement corresponds to a different stirrup width, such that the relative position of bracket members 120A and 120B and foot plate 40 are adjustable to vary the width of cast stirrup 100.

Second end portions 126A and 126B are adapted for attachment to adjustable side arm members 130A and 130B, respectively by screw attachment 39. Adjustable side arm members 130A and 130B each have two slots 121 aligned with a series of screw hole arrangements 138 located on second end portions 126A and 126B. Side arm members 130A and 130B can be moved relative to the second end portions 126A and 126B until the desired stirrup lengths/heights are achieved, at which point screws 39 are tightened.

Adjustable side arm members 130A and 130B each further comprise respective upper leg clamps 135A and 135B. In a similar manner, second end portions 126A and 126B of bracket members 120A and 120B also comprise respective lower leg clamps 125A and 125B.

FIG. 5 illustrates a step in the process of attaching cast stirrup 10 to a primary foot cast 55 (patient's leg not shown) using fiberglass overwrap 58.

An exemplary configuration of cast stirrup 10 assembled with primary foot cast 55 (patient leg not shown) and attachment casting material 58 is shown in FIG. 6.

It is obvious that the foregoing embodiments of the invention are examples and can be varied in many ways. Such present or future variations are not to be regarded as a departure from the spirit and scope of the invention, and all such modifications as would be obvious to one skilled in the art are intended to be included within the scope of the following claims.

1. A modular adjustable cast stirrup device comprising:
a first bracket member and a second bracket member, each of the bracket members being formed from a rigid strip having a horizontally oriented first end portion, a middle portion extending vertically upward from the first end portion, a second end portion, and an inwardly angled transition portion extending between the middle portion and the second end portion;
a first adjustable side arm member and a second adjustable side arm member, each of the adjustable side arm members comprising at least one leg clamp located on an inner side of each respective side arm member, wherein each of the adjustable side arm members is configured for removable and adjustable attachment to the second end portion of a respective bracket member;
a horizontally oriented foot plate member being configured for removable and adjustable attachment to the first end portion of each said bracket member;
wherein the relative position of the bracket member and the side arm member is adjustable to vary the length and/or height of the cast stirrup, and
wherein the relative position of the bracket member and the foot plate is adjustable to vary the width of the cast stirrup.

2. The cast stirrup device of claim 1, wherein the adjustable side arm member is attached to the bracket member by screw attachment.

3. The cast stirrup device of claim 2, wherein the adjustable side arm members comprise a series of screw hole arrangements configured to correspond to screw holes on the second end portions of the bracket members, wherein the relative position of the side arm members and the bracket members is adjustable to obtain a desired stirrup length/height.

4. The cast stirrup device of claim 2, wherein the adjustable side arm members comprise two slots aligned with series of screw hole located on the second end portions of the bracket members, wherein the side arm members are movable relative to the second end portions to obtain a desired stirrup length/height.

5. The cast stirrup device of claim 1, wherein the foot spacer plate is attached to the bracket member by screw attachment.

6. The cast stirrup device of claim 5, wherein the first end portion of the bracket member comprises a series of screw hole arrangements, each arrangement corresponding to a different stirrup width.

7. The cast stirrup device of claim 1, wherein the foot plate member comprises a foot pad.

8. The cast stirrup device of claim 1, wherein each of the bracket members comprises a leg clamp located on an inner side of the second end portion.

9. A process for assembling the cast stirrup as defined in claim 1, comprising the steps of:
affixing the foot plate to the bracket members by aligning the foot plate the horizontally oriented first end portion of the bracket members to provide a desired stirrup width and attaching the foot plate and bracket members using screw attachments; and
affixing the side arm members to the upper end of the bracket members by aligning the side arm members with the second end portion of the bracket members to provide a desired height of the assembled cast stirrup and attaching the side arm members and bracket members using screw attachments.

10. The process of claim 9, wherein the side arm members comprise a series of screw hole arrangements configured to correspond to screw holes on the second end potions of the bracket members, and wherein the process further comprises the step of aligning the screw holes on the side arm members with screw holes on the bracket members to obtain the desired stirrup length/height.

11. The process of claim 9, wherein the side arm members comprise two slots aligned with series of screw hole located on the second end portions of the bracket members, and wherein the process further comprises the step of sliding the side arm members relative to the bracket members to obtain the desired stirrup length/height.

12. A process for installing an assembled cast stirrup on a patient's casted leg, comprising the steps of:
placing the assembled stirrup over the patient's casted leg;
aligning the stirrup with a longitudinal axis of the patient's casted leg;
positioning the stirrup about 2 inches below a base of the cast and ensuring the base of the cast is parallel to the base of the stirrup;
applying a thin layer of soft roll over an upper portion of the stirrup including the leg clamps and side arm members;
applying a fiberglass cast material overwrap around the upper portion of the stirrup and cast to secure the stirrup in place; and
allowing the fiberglass overwrap to set.

13. The process of claim 12, wherein the cast stirrup is as defined in claim 1.

14. A kit for assembling a cast stirrup device comprising:
two bracket members, each said bracket members having a horizontally oriented first end portion, a middle portion extending vertically upward from the first end portion, a second end portion, and an inwardly angled transition portion extending between the middle portion and the second end portion, each of the bracket members comprising a leg clamp located on an inner side of the second end portion;
two adjustable side arm members, each of the adjustable side arm members comprising at least one leg clamp located on an inner side of each respective side arm member, wherein each of the adjustable side arm members is configured for removable and adjustable attachment to the second end portion of a respective bracket member;
a foot plate member, the foot plate member being configured for removable and adjustable attachment to the first end portion of each said bracket member;
a plurality of screw attachments;
instructions for assembly; and
optional tools for assembly.
